(19) United States
(12) Patent Application Publication
(10) Pub. No.: US 2017/0304133 A1
(43) Pub. Date: Oct. 26, 2017

(54) Limb Elevating Assembly

(71) Applicant: Angelica Jauregui, Cudahy, CA (US)

(72) Inventor: Angelica Jauregui, Cudahy, CA (US)

(21) Appl. No.: 15/137,468

(22) Filed: Apr. 25, 2016

(51) Int. Cl. A61G 7/057; A61F 5/37; A61F 5/32

(52) U.S. Cl. CPC A61G 7/057; A61F 5/32; A61F 5/3776

(57) ABSTRACT

A limb elevating assembly includes a strap that may be wrapped around a limb of a user. A cushion is coupled to the strap. The cushion is wrapped around itself when the strap is wrapped around the user's limb. Thus, the cushion elevates the user's limb on a support surface when the user lies on the support surface. The cushion inhibits bedsores from developing on the user's limb.

### BACKGROUND OF THE DISCLOSURE

### Field of the Disclosure

The disclosure relates to elevating devices and more particularly pertains to a new elevating device for elevating a bedridden user's limb above a bed thereby inhibiting bedsores from developing on the user's limb.

### SUMMARY OF THE DISCLOSURE

An embodiment of the disclosure meets the needs presented above by generally comprising a strap that may be wrapped around a limb of a user. A cushion is coupled to the strap. The cushion is wrapped around itself when the strap is wrapped around the user's limb. Thus, the cushion elevates the user's limb on a support surface when the user lies on the support surface. The cushion inhibits bedsores from developing on the user's limb.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top perspective view of a limb elevating assembly according to an embodiment of the disclosure.

FIG. 2 is a bottom view of an embodiment of the disclosure.

FIG. 3 is a top view of an embodiment of the disclosure.

FIG. 4 is a cross sectional view taken along line 4-4 of FIG. 3 of an embodiment of the disclosure.

FIG. 5 is a perspective in-use view of an embodiment of the disclosure.

### DESCRIPTION OF THE PREFERRED EMBODIMENT

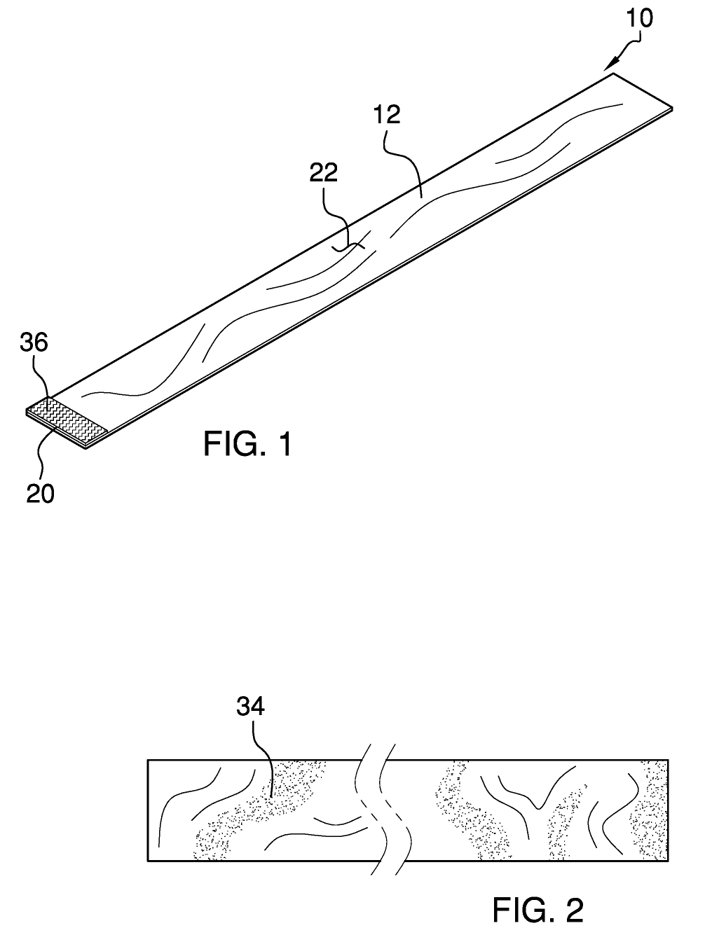

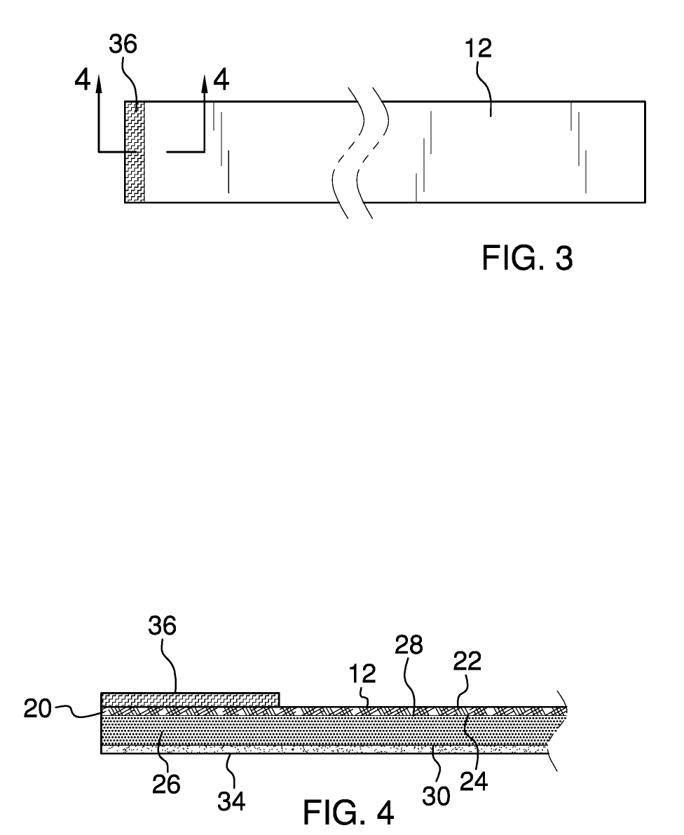

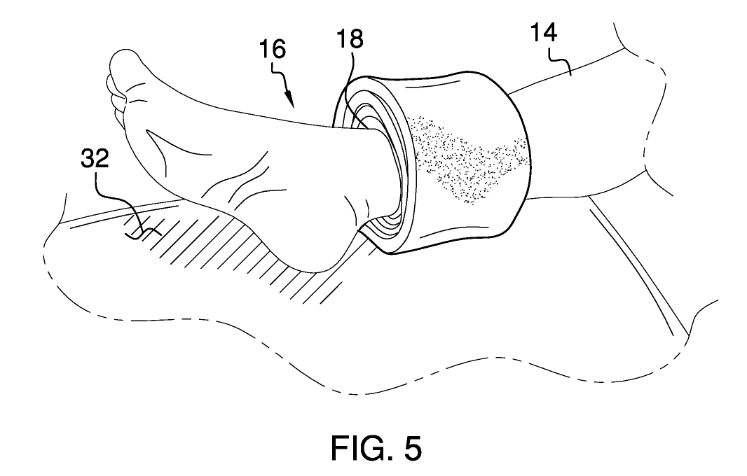

With reference now to the drawings, and in particular to FIGS. 1 through 5 thereof, a new elevating device embodying the principles and concepts of an embodiment of the disclosure and generally designated by the reference numeral 10 will be described.

As best illustrated in FIGS. 1 through 5, the limb elevating assembly 10 generally comprises a strap 12 that may be wrapped around a limb 14 of a user 16. The limb 14 may be the user's leg. The strap 12 may be positioned around an ankle 18 of the user's limb 14. The strap 12 has a first end 20, a top surface 22 and a bottom surface 24.

A cushion 26 is coupled to the strap 12. The cushion 26 has a first surface 28 and a second surface 30. The first surface 28 is coupled to the bottom surface 24 of the strap 12. The cushion 26 is coextensive with the strap 12. The cushion 26 is comprised of a resiliently compressible material. Thus, the cushion 26 enhances comfort of the strap 12 when the strap 12 is wrapped around the user's limb 14.

The cushion 26 is wrapped around itself when the strap 12 is wrapped around the user's limb 14. The cushion 26 elevates the user's limb 14 above a support surface 32 when the user 16 lies on the support surface 32. Thus, the cushion 26 inhibits bedsores from developing on the user's limb 14. The support surface 32 may be a bed or the like. The user 16 may be a bedridden user or the like.

A panel 34 is provided. The panel 34 is coupled to the second surface 30 of the cushion 26. Moreover, the panel 34 is coextensive with the cushion 26. The panel 34 may be comprised of a deformable material such as felt or the like.

A coupler 36 is provided and the coupler 36 is coupled to the strap 12. The coupler 36 is positioned on the top surface 22 of the strap 12. Additionally, the coupler 36 is aligned with the first end 20 of the strap 12. The coupler 36 is complementary with respect to the panel 34. Thus, the coupler 36 retains the strap 12 on the user's limb 14. The coupler 36 may comprise a hook and loop fastener or the like.

In use, the strap 12 is wrapped around the user's limb 14. The strap 12 is positioned adjacent to the user's ankle 18. The cushion 26 elevates the user's heel 38 from the support surface 32 when the user 16 lies on the support surface 32. Thus, the user's heel 38 is inhibited from developing bedsores resulting from prolonged contact with the support surface 32.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A limb elevating assembly being configured to be wrapped around a limb of a user thereby facilitating the limb to be elevated when the user lies down, said assembly comprising:
a strap being configured to be wrapped around a limb of a user; and
a cushion being coupled to said strap, said cushion being wrapped around itself when said strap is wrapped around the user's limb wherein said cushion is configured to elevate the user's limb on a support surface when the user lies on the support surface thereby inhibiting bedsores from developing on the user's limb.

2. The assembly according to claim 1, wherein:
said strap has a bottom surface; and
said cushion has a first surface and a second surface, said first surface being coupled to said bottom surface, said cushion being coextensive with said strap, said cushion being comprised of a resiliently compressible material wherein said cushion is configured to enhance comfort of said strap when said strap is wrapped around the user's limb.

3. The assembly according to claim 2, further comprising a panel being coupled to said second surface of said cushion, said panel being coextensive with said cushion.

4. The assembly according to claim 1, further comprising:
said strap having a top surface and a first end;
a panel being coupled to said strap; and
a coupler being coupled to said strap, said coupler being positioned on said top surface, said coupler being aligned with said first end, said coupler being complementary with respect to said panel wherein said coupler is configured to retain said strap on the user's limb.

5. A limb elevating assembly being configured to be wrapped around a limb of a user thereby facilitating the limb to be elevated when the user lies down, said assembly comprising:
a strap being configured to be wrapped around a limb of a user, said strap having a first end, a top surface and a bottom surface;
a cushion being coupled to said strap, said cushion having a first surface and a second surface, said first surface being coupled to said bottom surface, said cushion being coextensive with said strap, said cushion being comprised of a resiliently compressible material wherein said cushion is configured to enhance comfort of said strap when said strap is wrapped around the user's limb, said cushion being wrapped around itself when said strap is wrapped around the user's limb wherein said cushion is configured to elevate the user's limb on a support surface when the user lies on the support surface thereby inhibiting bedsores from developing on the user's limb;
a panel being coupled to said second surface of said cushion, said panel being coextensive with said cushion; and
a coupler being coupled to said strap, said coupler being positioned on said top surface, said coupler being aligned with said first end, said coupler being complementary with respect to said panel wherein said coupler is configured to retain said strap on the user's limb.
